(19) United States
(12) Patent Application Publication
(10) Pub. No.: US 2013/0095177 A1
(43) Pub. Date: Apr. 18, 2013

(54) METHOD OF PREPARING AN ORAL DOSAGE FORM COMPRISING FINGOLIMOD

(71) Applicants: Jana Paetz, Bonn (DE); Katrin Rimkus, Pullach (DE)

(72) Inventors: Jana Paetz, Bonn (DE); Katrin Rimkus, Pullach (DE)

(73) Assignee: RATIOPHARM GMBH, Ulm (DE)

(21) Appl. No.: 13/642,160

(22) PCT Filed: Apr. 21, 2011
(86) PCT No.: PCT/EP2011/002051

§ 371 Date: Dec. 23, 2012

(30) Foreign Application Priority Data
Apr. 22, 2010 (DE) 10 2010 017 944.2

(51) Int. Cl. A61K 31/137; A61K 9/20; A61J 3/02

(52) U.S. Cl. CPC A61K 31/137; A61J 3/02; A61K 9/2095
USPC 424/452; 514/653; 424/400; 424/464; 424/451; 424/465

(57) ABSTRACT

The present invention relates to a method of preparing an intermediate containing fingolimod, a method of preparing granules containing fingolimod, a method of preparing an oral dosage form containing fingolimod and accordingly intermediates, granules and oral dosage forms obtainable by that method.

The present invention relates to a method of preparing an intermediate containing fingolimod, a method of preparing granules containing fingolimod, a method of preparing an oral dosage form containing fingolimod and accordingly intermediates, granules and oral dosage forms obtainable by that method.

Fingolimod, which is also referred to as “FTY720”, is a synthetic imitation of myriocin, a metabolic product of the fungus Isaria sinclairii. Fingolimod is a modulator of the sphingosine-1 phosphate receptor, which, after phosphorylation, can bind sphingosine-1 phosphate receptors, especially of T and B-lymphocytes. This inhibits the migration of lymphocytes from the lymph nodes into the blood and hence reduces their distribution in the central nervous system. Inflammatory T-lymphocytes are possible triggers for the destruction of the neural myelin sheaths, which are responsible for the typical symptoms of multiple sclerosis. For this reason, fingolimod is a possible means for the treatment of multiple sclerosis and especially for the treatment of patients with relapsing-remitting multiple sclerosis.

The IUPAC name of fingolimod is 2-amino-2-(2-[4-octylphenyl]ethyl)-1.3-propane diol. The chemical structure of fingolimod is shown in formula (1) below:

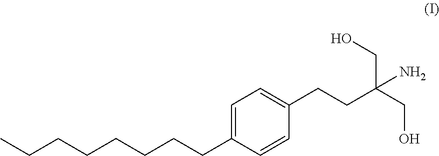 [SMILES: CCCCCCCCc1ccc(CCC(N)(CO)CO)cc1]

The synthesis of fingolimod is described in, for example, the European patent application EP 0 627 406.

Fingolimod is currently undergoing Phase III clinical trials, in which doses of 0.5 and 1.25 mg are being administered orally once a day. For the treatment of multiple sclerosis, doses ranging from 0.25 to 2.5 mg, i.e. very small amounts, are generally contemplated.

The proportion of the active agent in the total weight of the formulation (incl. active agent), or the formulation unit, especially in the case of formulations for oral administration, is typically in the range of only a few per cent by weight, such as 0.25 to 4% by weight. During preparation of the formulation, this small proportion of active agent can lead to considerable problems with regard to the uniformity of the content of active agent in the individual formulation units. For example, minor changes in the content of active agent, perhaps caused by changes in the flowability, especially of the active agent, and/or separation phenomena, can lead to major variations.

The Ph. Eur. 6.0 section 2.9.6 therefore prescribes a uniformity test for the content of active agent in formulation units. According to that test, each individual content of 10 units must lie between 85 and 115 per cent of the average content. If more than one individual content lies outside that limit or if one individual content lies outside the limits of 75 to 125 per cent of the average content, the formulation units do not pass the test.

One problem to be solved by the present invention therefore consists in providing a method making it possible to prepare an oral dosage form containing fingolimod which exhibits good uniformity (homogeneity) of the content of active agent, and also in providing such a dosage form.

A further problem of the present invention consists in providing an oral dosage form of fingolimod which exhibits good storage stability with regard to the uniformity of the content of active agent. A further problem of the present invention consists in providing an oral dosage form containing fingolimod whose content of active agent, especially also after a lengthy storage time, lies within the concentration limits of 85 and 115 per cent and preferably 90 and 110 per cent of the average content according to Ph. Eur.

It has unexpectedly been possible to solve the above-mentioned problems by a method of preparing an intermediate comprising fingolimod (a) and one or more excipients (b), the particle sizes of which are within a specific range, a corresponding intermediate, granules and an oral dosage form containing the intermediate, and also a method of preparing it.

One subject matter of the present invention is a method of preparing an intermediate comprising (a) fingolimod and (b) one or more pharmaceutically acceptable excipients, wherein the method comprises the following steps:
(i) optionally mixing (a) fingolimod and (b) the excipient or the plurality of excipients,
(ii) jointly comminuting (a) fingolimod and (b) the one or more excipients into intermediate particles such that 90 per cent by volume of all the resulting intermediate particles have a particle size of less than 250 μm and greater than 0.6 μm.

A further subject matter of the invention is an intermediate which is obtainable by means of the method of the invention of preparing an intermediate. Another subject matter of the invention is accordingly an intermediate comprising particles of (a) fingolimod and (b) one or more pharmaceutically acceptable excipients, wherein 90 per cent by volume of the particles have a particle size of less than 250 μm and greater than 0.6 μm.

A further subject matter of the invention relates to granules containing (a) fingolimod and (b) one or more pharmaceutically acceptable excipients, which are obtainable by the method of the invention of preparing an intermediate and by the following step:
(iii) granulating the intermediate and optionally one or more additional pharmaceutical excipients.

A further subject matter of the invention is a method of preparing an oral dosage form containing (a) fingolimod and (b) one or more pharmaceutically acceptable excipients, comprising the method of the invention of preparing an intermediate and by the following steps:
(iii) optionally further processing the intermediate, optionally with the addition of one or more additional pharmaceutically acceptable excipients, preferably by granulation, spray-drying or lyophilisation, into an intermediate product,
(iv) compressing the intermediate from step (ii) or the intermediate product from step (iii) and optionally one or more additional pharmaceutically acceptable excipients into tablets.
   or
   filling the intermediate from step (ii) or the intermediate product from step (iii) and optionally one or more additional pharmaceutically acceptable excipients into capsules or sachets or other suitable containers.

An oral dosage form containing (a) fingolimod and (b) one or more pharmaceutically acceptable excipients which is obtainable by this method is likewise a subject matter of the invention. An oral dosage form containing the intermediate or granules of the invention is accordingly also a subject matter of the invention. Finally, intermediates, granules or oral dosage forms for the treatment of multiple sclerosis, preferably relapsing-remitting multiple sclerosis, are also part of the present invention.

It has surprisingly transpired that intermediates in the particle size range specified above are particularly advantageous for further use or further processing and that, as a result, a uniform content of active agent, especially in the oral dosage forms based on them, can be achieved. Using intermediates with particles in this size range means that no unwanted agglomeration or separation phenomena occur during the further use or further processing, especially into oral dosage forms, or do not occur to any considerable extent.

In conventional formulations, the irregularity of the shape and size of individual particles or crystals, which is typical for fingolimod, in combination with the very small amounts of active agent, led to major problems in the uniformity of content of the active agent. This applied especially when compressing fingolimod into tablets and other volume-metered processes, in which the particle characteristics play a particularly noticeable role.

The advantages of the method, an essential step of which is the joint comminution of the active agent and excipient, and of the resulting intermediate are particularly surprising in view of the fact that fingolimod has a low melting point. The melting point of the hydrochloride salt, for example, is only about 102 to 107° C. With such a low melting point, there is normally a risk that any reduction in size and especially mechanical comminution processes entail various disadvantages: an increase in the surface area usually reduces the stability of the active agent. An increased degradation profile may also occur. In addition, with comminution processes there is a risk of thermal stress and partial amorphisation, which in turn leads to an increase in the hygroscopic properties and thus in agglomeration. These phenomena can lead to a reduction in the storage stability and inadequate uniformity of the content of active agent (content uniformity). Furthermore, especially with polymorphous substances, comminution processes can lead to undesirable recrystallisation phenomena, which may result in different solubilities, for example. It has nevertheless surprisingly been found that these possible disadvantages do not occur with the method of the invention or with the resulting intermediate. Consequently, in the context of the method of the invention, fingolimod is preferably not dissolved in a solvent at any time during the method, but is merely suspended or wetted in any process steps that might possibly involve solvents or dispersants.

In the context of the present invention, the term “fingolimod” comprises 2-amino-2-(244-octylphenyl]ethyl)-1,3-propanediol according to the above formula (I). In addition, the term “fingolimod” comprises all the pharmaceutically acceptable salts, hydrates and/or solvates thereof. Acid addition salts are the salts preferably used. Examples of suitable salts are hydrochlorides, carbonates, hydrogen carbonates, acetates, lactates, butyrates, propionates, sulphates, methane sulphonates, citrates, tartrates, nitrates, sulphonates, oxalates and/or succinates. Fingolimod hydrochloride is particularly preferably used.

For all the embodiments of this invention, the term “fingolimod” preferably means fingolimod in crystalline form, i.e. preferably more than 90% by weight of the fingolimod used is present in crystalline form, and particularly preferably 100% by weight of the fingolimod used is present in crystalline form.

In the context of this invention, fingolimod (a) is preferably used as the sole active agent. The intermediate of the present invention accordingly contains preferably no further active agent. Embodiments with one or more further active agents are, however, also conceivable.

In a preferred embodiment, the fingolimod per se or a pharmaceutically acceptable salt thereof used in the dosage form has a water content of 0.01 to 10% by weight, more preferably 0.25 to 8.0% by weight, e.g. 0.27 to 7.5% by weight and particularly preferably 0.29 to 5% by weight. In the context of this application, the water content is preferably determined according to the Karl Fischer method, using a coulometer at 160° C. A Metrohm 831 KF coulometer with a titration cell without a diaphragm is preferably used. Usually, a 20 mg sample of fingolimod is analysed.

According to the present invention, an “intermediate” is preferably understood to mean a pharmaceutical composition which is not administered directly, but is instead converted into an applicable oral dosage form by means of suitable methods, such as granulation and/or compression,.

In the method of the invention, there may be an optional step involving (i) mixing (a) fingolimod and (b) the excipient or the plurality of excipients, which is performed before their joint comminution (ii).

In the following, for the sake of simplicity, reference will generally be made only to “(b) excipients”, even though the term is intended also to encompass embodiments in which only one excipient is used.

“Mixing” is to be understood in the context of the present invention as meaning a process of combining substances with the aim of achieving a substantially homogeneous distribution of different substances by the effect of mechanical forces. Mixing for the purposes of the invention is performed in conventional mixing devices, such as roll mixers, shaking mixers, free-fall mixers, shear mixers, ploughshare mixers, planetary mixing kneaders, Z or sigma kneaders or fluid or intensive mixers. A free-fall mixer is preferably used.

The time for the optional step of mixing (i) may, for example, be 0.5 minutes to 1 hour, preferably 2 minutes to 50 minutes, more preferably 5 minutes to 30 minutes.

Alternatively, the homogeneous distribution of (a) fingolimod and (b) excipients is effected in the context of step (ii), joint comminution.

The particle size distribution achieved by the comminution indicates that the comminution step in the present invention is not just concerned with separating the agglomerates or the like which usually occur in powders, but rather with a systematic means of adjusting a specific particle size range.

According to a preferred embodiment of the invention the (ii) joint comminution of (a) fingolimod and (b) excipients to intermediate particles comprises the following (part-)steps:
(ii) (1) joint comminution of (a) fingolimod and a first part of the excipients (b);
(2) addition of a second part of the excipients (b) and joint comminution of (a) fingolimod and the first and second parts of the excipients (b).

Analogously, further steps of addition and comminution can follow, depending on how many parts the excipients (b) are divided into. Each step or part-step may be preceded by a mixing step, i.e. an addition step can be accompanied in each case by a mixing step, for example.

“A part” can refer to a proportion of the amount expressed in terms of weight. By way of example, half the total amount of excipients (b) by weight can be comminuted to begin with in a first step (1) with fingolimod (a), then in a second step (2) the remaining second half is added, followed once again by joint comminution. Similarly, in the case of a division into thirds, two additions (in steps (2) and (3)) and hence a third step (3) following the second step (2) are necessary. In the case of a division into quarters, a total of 4 analogous steps are needed, etc. By way of example, fingolimod is preferably first comminuted with 10 to 50% by weight of the excipients (b). After that, the remainder of the excipients (b) is added in one to three stages, for example.

In a further embodiment, “a part” may also refer to a type of excipient or excipients. In step (1), for example, there may be a joint comminution of (a) fingolimod and a first excipient (b) or a first group of excipients (b), and a second step (2) may involve the addition of a second excipient (b) or a second group of excipients (b) and the joint comminution of (a) fingolimod and the first and second excipients or groups of excipients (b). The first and second excipients, or first and second groups of excipients, are to be understood in this context as being different from one another in each case.

“A part” can also comprise a combination of the amount and type. In a preferred embodiment of the invention, the (ii) joint comminution of (a) fingolimod and (b) excipients to intermediate particles may accordingly comprise the following (part-)steps:
(ii) (1) joint comminution of (a) fingolimod and a first quantitative part of a total amount of a first excipient;
   (2) addition of a second excipient and joint comminution of (a) fingolimod and the first and second excipients;
   (3) addition of a second quantitative part of the total amount of the first excipient and joint comminution of (a) fingolimod and the first and second excipients.

If the second quantitative part of the first excipient in part-step (3) does not correspond to the difference between the first quantitative part and the total amount, corresponding further steps analogous to step (3) may follow. The second excipient can accordingly also be added successively, divided up into a number of quantitative parts, e.g. alternating with the first excipient.

Various methods can be used for jointly comminuting (a) fingolimod and (b) excipients into intermediate particles.

In a preferred embodiment of the method of preparing an intermediate, step (ii) of jointly comminuting (a) fingolimod and (b) excipients comprises joint milling.

In general, “milling” is understood to mean the comminution of substances, especially active agents and excipients, to a predetermined particle size spectrum by applying an external force. The comminution principle can conventionally involve the effect of pressure, friction, cutting, impingement, impact, or combinations thereof.

The joint milling of (a) fingolimod and (b) excipients has the advantage that (a) fingolimod and (b) excipients are distributed particularly homogeneously. This effect can be further promoted by adding the excipients (b) in stages, as described above. Furthermore, an appropriate choice of excipient can make it posbible to deposit the active agent on the excipient. This is particularly advantageous in achieving the desired uniformity in the intermediate, granules and oral dosage form. For this purpose, it is particularly advantageous to use excipients with a large surface area, such as with a surface area of at least 0.5 m²/g, e.g. at least 1 m²/g, 1.5 m²/g, 2 m²/g or particularly preferably 2.5 m²/g. The surface area is preferably determined in this connection by means of BET measurement. Examples of suitable excipients are, for example, fillers, surface stabilisers and wetting agents, as described below. In the context of this invention, milling can mean both wet milling and also dry milling. In both milling processes, it must be ensured that the milling temperature remains within a range of up to 50° C., more preferably up to 40° C.

In the present context, “dry milling” is understood to mean the comminution of solids in the absence of solvents. In dry milling, the addition of one or more excipients in stages, as explained above, is particularly advantageous.

In the present context, “wet milling” is understood to mean the comminution of solids in a liquid phase. The liquid phase here is preferably a liquid in which (a) fingolimod and (b) excipients do not dissolve, or not significantly. Examples of suitable milling fluids are methanol, ethanol, isopropanol, acetone, chloroform, butanol, ethyl acetate, heptane, pentanol or mixtures thereof. Acetone or chloroform is preferably used. Wet milling in combination with subsequent drying is a preferred embodiment of the comminution step (ii). The drying can be performed with, for example, one or more of the following methods: spray-drying, vacuum drying, freeze drying etc.

In one embodiment of this invention, the intermediate is produced by wet milling followed by drying. For this purpose, spray-drying is preferably used as the drying step.

In one embodiment, in which the joint comminution is performed by milling using a milling liquid (wet milling), an additional excipient can be added to the milling liquid, which dissolves in the milling liquid. The excipient then preferably serves to increase the viscosity of the milling liquid. It is preferably intended to prevent any unwanted agglomeration and shearing phenomena and thus to achieve a more efficient wet milling process. The excipient here is preferably selected such that it combines homogeneously with the active agent during a subsequent drying step. In other embodiments, it may also be advantageous to use an excipient which is not soluble in the milling liquid. The intermediate obtained in this way is particularly good at preventing any separation and thus promotes particularly good uniformity in the intermediate and granules produced from it, or an oral dosage form produced from it. Suitable conceivable excipients are, for example, HPMC, PVP or sodium lauryl sulphate. The milling is generally performed in conventional milling apparatuses, such as in a ball mill, air jet mill, pin mill, classifier mill, cross beater mill, disk mill, edge mill, mortar grinder, rotor mill, rolling crusher or hammer mill.

The milling time is usually 0.5 minutes to 2 hours, preferably 2 minutes to 60 minutes, more preferably 5 minutes to 50 minutes.

The milling and, where applicable, mixing conditions are selected in accordance with the invention such that an intermediate is obtained with the particle size distribution of the invention described above. According to the invention, 90 per cent by volume of all the intermediate particles have a particle size of less than 250 μm and greater than 0.6 μm. For example, 90 per cent by volume of all the intermediate particles may have a particle size in one of the following preferred ranges: 1 μm to 200 μm, 2 μm to 180 μm, 3 μm to 170 μm, 5 μm to 100 μm, 7 μm to 80 μm or 10 μm to 50 μm.

The D₉₀ value of the intermediate of the invention may in this case possess one of the following values:

D₉₀<250 μm

D₉₀<200 μm;

D₉₀<180 μm;

D₉₀<170 μm;

D₉₀<100 μm;

D₉₀<80 μm;

D₉₀<50 μm.

This means that the present invention also encompasses fingolimod particles in the size ranges mentioned above for the intermediate particles, i.e., for example, fingolimod particles of which 90 per cent by volume have a size between 0.6 μm and 250 μm, 1 μm and 200 μm; 2 μm and 180 μm; 3 μm and 170 μm; 5 μm and 100 μm; 7 μm and 80 μm or 10 μm and 50 μm, such as in combination with one or more of the corresponding D₉₀ values mentioned above for the intermediate particles.

The “particle size” of a particle to be determined is understood for the purposes of the invention to mean the diameter of an equivalent particle which is assumed to be spherical and to have the same light-scattering pattern as the particles to be determined. In accordance with the invention, the particle size is determined by means of laser diffractometry. In particular, a Malvern Instruments Mastersizer 2000 is used to determine the particle size. Wet measurement with a dispersion of particles in dispersant, 2,000 rpm, ultrasound 60 seconds with a shading of 4 to 15% is preferable. The evaluation is carried out for particles with a D₅₀ value of less than 5.0 μm using the Mie method and for particles with a D₅₀ value of at least 5.0 μm using the Fraunhofer method.

The terms “particles of the intermediate” and “intermediate particles” are used synonymously herein.

“Particle size distribution of the intermediate” is to be understood in the context of this invention as meaning the statistical distribution of the volume portions based on all the particle sizes of the particles of the intermediate. “Volume portion” in the present case means the volume-based proportion in per cent of all particles with a defined particle size.

The D₉₀ value of the particle size distribution of the intermediate describes the particle size at which 90% by volume of the particles have a smaller particle size than the particle size corresponding to the D₉₀ value.

Similarly, the D₅₀ value of the particle size distribution is defined as the particle size at which 50% by volume of the particles have a smaller particle size than the particle size corresponding to the D₅₀ value. Likewise, 50% by volume of the particles then have a larger particle size than the D₅₀ value.

Analogously, the D₁₀ value of the particle size distribution of the intermediate is defined as the particle size at which 10% by volume of the particles have a smaller particle size than the particle size corresponding to the D₁₀ value.

In addition, the joint comminution (ii) can be carried out in further embodiments in such a way that 50 per cent by volume of all the resulting intermediate particles have a particle size of 80 μm or less, e.g.:

D₅₀≦50μm, or

D₅₀≦40μm, or

D₅₀≦30μm

The breadth of the particle size distribution is preferably relatively narrow, i.e. the particle sizes of the intermediate particles lie in a relatively narrow range. In one embodiment, the joint comminution (ii) can accordingly be carried out in such a way that the particle sizes of 90 per cent by volume of all the resulting intermediate particles (D₉₀), the particle sizes of 50 per cent by volume of all the resulting intermediate particles (D₅₀) and the particle sizes of 10 per cent by volume of all the resulting intermediate particles (D₁₀) satisfy the following relationship:

(D₉₀−D₁₀)/D₅₀≦7.0,

preferably 0.5≦(D₉₀−D₁₀)/D₅₀≦4.0, more preferably 1.0≦(D₉₀−D₁₀)/D₅₀≦3.1.

In alternative embodiments, the following relationships, for example, may be satisfied:

(D₉₀−D₁₀)/D₅₀≦7.0;

(D₉₀−D₁₀)/D₅₀≦3.1;

(D₉₀−D₁₀)/D₅₀≦2.5;

(D₉₀−D₁₀)/D₅₀≦2.0;

(D₉₀−D₁₀/ )D₅₀≦1.65;

(D₉₀−D₁₀)/D₅₀≦1.2;

(D₉₀−D₁₀)/D₅₀≦0.75; or

(D₉₀−D₁₀)/D₅₀≦0.5.

Additionally, the joint comminution (ii) can preferably be carried out in such a way that the resulting intermediate particles have a monomodal particle size distribution. In an alternative embodiment, the particles of fingolimod (a) or the particles of active agent (b) each have a monomodal particle size distribution in their own right. “Monomodal” is in this case understood to mean that the particle size distribution only has one maximum when represented in a histogram and/or a frequency distribution curve.

In addition, the mixing and milling conditions in the method of the invention are preferably selected such that an intermediate with a uniformity of the mixture of 90% to 110%, more preferably 92% to 108%, even more preferably 94% to 106%, particularly preferably 96% to 104% and especially 98% to 102%, is obtained. The “uniformity of the mixture” refers here to the uniformity of the content of active agent in different intermediate samples. In order to determine the uniformity of the mixture, 20 individual samples with a volume of 10 ml each are taken from the intermediate at random. The uniformity of the content of active agent is then determined in accordance with Ph. Eur. 6.0, Chapter 2.9.6, HPLC being used as the analytical process. This means that each of twenty individual samples of the intermediate has a fingolimod content of between 90% and 110%, preferably 92% to 108%, even more preferably 94% to 106%, particularly preferably 96% to 104% and especially 98% to 102% of the average content of those twenty individual samples. An intermediate with such uniformity is accordingly an embodiment of the present invention.

In the context of the present invention, the term “excipient” (b) encompasses:

fillers (b1), surface stabilisers (b2), disintegrants (b3), flow conditioning agents (b4) and/or lubricants (b5). Where appropriate, wetting agents (b6) can also be used as excipients.

Of the above-mentioned components, the intermediate of the invention contains, for example, (a) and at least one excipient from the group of fillers (b1), surface stabilisers (b2), flow conditioning agents (b4) and wetting agents (b6). When a wet milling process is used, the intermediate preferably contains, for example, (a) and (b1), (b2), and (b6) of the above-mentioned components. When a wet milling process is used, the intermediate preferably contains at least a filler (b1) and a surface stabiliser (b2).

Fillers (b1) may, for example, be used in amounts between 10 and 99% by weight, preferably between 25 and 97% by weight, and particularly preferably between 30 and 95% by weight, based on the total weight of the intermediate.

Surface stabilisers (b2) are used, for example, in amounts of 1 to 30% by weight, preferably 2 to 20% by weight, particularly preferably 3 to 15% by weight, based on the total weight of the intermediate.

Flow conditioning agents (b4) may, for example, be used in amounts of 0.1 to 10% by weight, preferably 0.5 to 5% by weight, particularly preferably 1 to 3% by weight, based on the total weight of the intermediate.

Wetting agents (b6) may, for example, be used in amounts of 0.001 to 1.0% by weight, preferably 0.01 to 0.5% by weight, more preferably 0.015 to 0.15% by weight, particularly preferably 0.02 to 0.1% by weight, based on the total weight of the intermediate.

In a preferred embodiment, the intermediate of the invention accordingly contains
(a) fingolimod between 0.1 and 25% by weight, preferably between 0.15 and 15% by weight, particularly preferably between 0.2 and 5% by weight,
(b1) fillers between 10 and 99% by weight, preferably between 25 and 97% by weight, particularly preferably between 30 and 95% by weight,
(b2) surface stabiliser between 1 and 30% by weight, preferably 2 to 20% by weight, particularly preferably 3 to 15% by weight,
(b4) flow conditioning agent(s) between 0.1 to 10% by weight, preferably 0.5 to 5% by weight, particularly preferably 1 to 3% by weight, and/or
(b6) wetting agent(s) between 0.001 and 1.0% by weight, preferably 0.01 to 0.5% by weight, more preferably 0.015 to 0.15% by weight, particularly preferably 0.02 to 0.1% by weight,
   based on the total weight of the intermediate.

The expression “total weight of the intermediate” refers in this context to the weight of the active agent and excipients contained in the intermediate. In other words, it refers to the weight of the intermediate without solvents (used, for example, in the wet milling process described above). The same applies, mutatis mutandis, to the granules and the oral dosage form.

It is particularly preferable for especially the excipients (b3) and (b5) only to be added to the intermediate in the context of further processing, such as before or during a granulation and/or compression step described below. It is accordingly preferred for the intermediate to contain no disintegrant (b3) and/or no lubricant (b5), preferably neither. Excipients (b1), (b2), (b4) and/or (b6) can likewise only be added to the intermediate, proportionately or additionally where applicable, in the context of further processing or use. In this context, it is, for example, possible for the excipients (b) optionally added before or during a further processing step, such as a granulation step and/or compression step, likewise to have the D₁₀, D₅₀ and/or D₉₀ values for the particle size distribution explained above for the intermediate.

The granules, lyophilisate or intermediate product of the invention obtained by a different kind of further processing, e.g. spray-drying, and the oral dosage form of the invention may contain filler (b1), surface stabiliser (b2), disintegrant (b3), flow conditioning agent (b4), lubricant (b5) and/or wetting agent (b6) as excipients, possibly in addition to the excipients of these categories already contained in the intermediate.

In a preferred embodiment, the oral dosage form, especially the tablet, preferably contains disintegrant (b3) and/or lubricant (b5) in addition to the intermediate. In addition to the excipients (b) contained in the intermediate, the granules, the lyophilisate or the intermediate product obtained by a different kind of further processing, or the oral dosage form may also contain pharmaceutically acceptable excipients of the same category (b1) to (b6) and/or additional amounts of the excipients (b) contained in the intermediate.

Disintegrants (b3) are used, for example, in amounts of up to 30.0% by weight, such as 0 to 25.0% by weight, preferably 1.0 to 20.0% by weight, particularly preferably 3.0 to 15.0% by weight, based on the total weight of the oral dosage form.

Lubricants (b5) are used, for example, in amounts of up to 10% by weight, such as 0.1 to 5.0% by weight, preferably 0.2 to 2.0% by weight, particularly preferably 0.5 to 1.5% by weight, based on the total weight of the oral dosage form.

In a preferred embodiment, the oral dosage form of the invention, especially the tablet of the invention, contains
(a) fingolimod between 0.1 and 4.0% by weight, such as between 0.1 and 2.5% by weight, preferably between 0.15 and 1.5% by weight, particularly preferably between 0.2 and 1.2% by weight,
(b1) fillers between 30.0 and 99.8% by weight, preferably between 55.0 and 98.0% by weight, particularly preferably between 75.0 and 95.0% by weight,
(b2) surface stabiliser between 0.1 and 30.0% by weight, preferably 0.3 to 15.0% by weight, particularly preferably 0.5 to 10.0% by weight,
(b3) disintegrant between 0 and 30.0% by weight, preferably 1.0 to 20.0% by weight, particularly preferably 3.0 to 15.0% by weight,
(b4) flow conditioning agent between 0 and 10.0% by weight, preferably 0.1 to 6.0% by weight, particularly preferably 0.8 to 4.0% by weight,
(b5) lubricant between 0 and 10.0% by weight, preferably 0.1 to 5.0% by weight, particularly preferably 0.5 to 3.0% by weight,
(b6) wetting agent between 0 and 1% by weight, preferably 0.0015 to 0.75% by weight, particularly preferably 0.0025 to 0.5% by weight,
   based on the total weight of the (non-film-coated) oral dosage form, preferably the tablet.

In the context of this invention, “fillers” (b₁) are understood to mean substances which are usually described as pharmaceutical fillers or filling agents and can also be referred to as constituents, extenders or basic materials. These fillers are typically substances which are needed in order to form the body, or mass, of the oral dosage form in the case of dosage forms with small amounts of active agent, so as to obtain a sufficient amount of dosage form mass for a suitable dosage form size.

Fillers for the purposes of the invention are, for example: lactose, lactose derivatives, starch, starch derivatives, treated starch, chitin; cellulose and derivatives thereof, e.g. microcrystalline cellulose (e.g. Avicel®), calcium phosphates, such as calcium hydrogen orthophosphate, especially in the form of the dihydrate sucrose, calcium carbonate, magnesium carbonate, magnesium oxide, maltodextrin, calcium sulphate, dextrates, dextrin, dextrose, hydrogenated vegetable oil, kaolin, sodium chloride, potassium chloride and mixtures thereof can be used. Similarly, SiO₂ modified (silicified) microcrystalline cellulose (e.g. Prosolv®, Rettenmaier & Söhne, Germany) can be used.

Other fillers that can be used are sugar alcohols and/or disaccharides, such as mannitol, sorbitol, xylitol, isomalt, glucose, fructose, maltose and mixtures thereof. The term “sugar alcohols” in this context also includes monosaccharides.

The fillers, especially in the intermediate, are preferably selected from sucrose, microcrystalline cellulose, silicified microcrystalline cellulose, lactose, calcium hydrogen orthophosphate dihydrate and starch.

The intermediate of the invention (and correspondingly the intermediate product and oral dosage form obtained from it) preferably contains not only fillers, but also surface stabilisers (b2). In general, surface stabilisers (b2) are understood to mean substances which can prevent the reagglomeration of particles, especially milled particles. The surface stabiliser is preferably a polymer. In addition, the surface stabiliser also includes substances which behave like polymers. Examples of these are fats and waxes. They also include low-molecular-weight oligomers, natural polymers or emulsifiers. Preferred surface stabilisers contain non-ionic or ionic emulsifiers.

The surface stabiliser (b2) may be hydrophilic polymers. This refers to polymers which possess hydrophilic groups. Examples of suitable hydrophilic groups are hydroxy, amino, carboxy, sulphonate. In addition, the hydrophilic polymer which can be used in order to prepare the intermediate preferably has a weight-average molecular weight of 1,000 to 150,000 g/mol, more preferably 2,000 to 90,000 g/mol. The weight-average molecular weight is preferably determined in the context of this application by means of gel permeation chromatography.

The intermediate of the invention may, for example, comprise one or more of the following hydrophilic polymers as surface stabiliser: polysaccharides, such as hydroxypropyl methyl cellulose (HPMC), methyl cellulose, hydroxyethyl cellulose, ethyl hydroxyethyl cellulose, hydroxypropyl cellulose (HPC), salts of carboxymethyl cellulose; polyvinyl pyrrolidone (e.g. PVP 25), polyvinyl alcohol, polymers of acrylic acid and their salts, polyacrylamide, polymethacrylates, vinyl pyrrolidone/vinyl acetate copolymers (such as Kollidon® VA64, BASF), polyalkylene glycols and their derivatives, such as polypropylene glycol or preferably polyethylene glycol, polyethylene sorbitan fatty acid ester, co-block polymers of polyethylene glycol, especially co-block polymers of polyethylene glycol and polypropylene glycol (Pluronic®, BASF), and mixtures of the polymers mentioned.

The surface stabilisers preferably used are polyvinyl pyrrolidone, preferably with a weight-average molecular weight of 10,000 to 60,000 g/mol, especially 12,000 to 40,000 g/mol, copolymer of vinyl pyrrolidone and vinyl acetate, especially with a weight-average molecular weight of 45,000 to 75,000 g/mol and/or polymers of acrylic acid and their salts, especially with a weight-average molecular weight of 50,000 to 250,000 g/mol. In addition, HPMC is preferably used, especially with a weight-average molecular weight of 20,000 to 90,000 g/mol and/or preferably a proportion of methyl groups of 10 to 35% and a proportion of hydroxy groups of 1 to 35%. Likewise, HPC is preferably used, especially with a weight-average molecular weight of 50,000 to 100,000 g/mol. Also, polyethylene glycol with a number-average molecular weight of 2,000 to 40,000 g/mol, especially from 3,500 to 25,000 g/mol, is preferably used. Likewise, a polyethylene/polyethylene block copolymer is preferably used, wherein the polyethylene content is preferably 70 to 90% by weight. The polyethylene/polyethylene block copolymer preferably has a number-average molecular weight of 1,000 to 30,000 g/mol, more preferably from 3,000 to 15,000 g/mol.

Further examples of natural surface stabilisers are gelatine, casein, lecithin, dextran, gum arabic, gum traganth and/or cholesterol. Fatty acids and their derivatives and salts, sorbitan esters and silicates can also be used.

When the polymer used as the surface stabiliser (b2) is dissolved in water in an amount of 2% by weight, the resulting solution preferably has a viscosity of 0.1 to 25 mPa×s, more preferably 1.0 to 18 mPa×s, especially 2 to 15 mPa×s, measured at 25° and determined in accordance with Ph. Eur., 6th edition, Chapter 2.2.10. Especially in the case of HPMC, the resulting solution preferably has a viscosity of 2 to 10 mPa×s.

In the context of this invention, it is also possible to use any mixtures of the above-mentioned surface stabilisers.

In the context of the present invention, especially in the intermediate, it is particularly advantageous especially to use surface stabilisers (b2) and/or fillers (b1) with low brittleness.

Surface stabilisers can generally be classified with reference to the change in the shape of the particles under compression pressure (compaction): Plastic excipients are characterised by plastic deformation, whereas when compressive force is exerted on brittle excipients, the particles tend to break into smaller particles. Brittle behaviour on the part of the surface stabiliser can be quantified by the increase in the surface area in a moulding. In the art, it is customary to classify the brittleness in terms of the “yield pressure”. According to a simple classification, the values for the “yield pressure” here are low for plastic substances but high in the case of friable substances, on the other hand [Duberg, M., Nyström, C., 1982, Studies on direct compression of tablets VI. Evaluation of methods for the estimation of particle fragmentation during compaction. Acta Pharm. Suec. 19, 421-436; Humbert-Droz P., Mordier D., Doelker E. Méthode rapide de détermination du comportement à la compression pour des études de préformulation. Pharm. Acta Helv., 57, 136-143 (1982)). The “yield pressure” describes the tension that has to be reached for the substance (i.e. the surface stabiliser) to begin to flow plastically.

The “yield pressure” is preferably calculated using the reciprocal of the gradient of the Heckel plot, as described in York, P., Drug Dev. Ind. Pharm. 18, 677 (1992). The measurement here is preferably carried out at 25° C. and a deformation rate of 0.1 mm/s. In the context of the present invention, a surface stabiliser is deemed a non-brittle surface stabiliser if it has a “yield pressure” of no more than 150 MPa, preferably 5 to 80 MPa.

Examples of preferred non-brittle excipients are HPMC and polyvinyl pyrrolidone, preferably with the above-mentioned molecular weights.

“Disintegrants” (b3) is the term used herein to describe substances which accelerate the disintegration of a dosage form, especially a tablet, after it is placed in water. Suitable disintegrants are, for example, organic disintegrants such as carrageenan, croscarmellose and/or crospovidone (such as Kollidon® CL). Alkaline disintegrants can likewise be used. The term “alkaline disintegrants” means disintegrants which, when dissolved in water, produce a pH level of more than 7.0. Croscarmellose or crospovidone are preferred.

The task of flow conditioning agents (b4) is to reduce both the interparticular friction (cohesion) between the individual particles in a tableting mixture and their adherence to the wall surfaces of the press mould (adhesion). An example of an additive to improve powder flowability is disperse, or colloidal, silica (e.g. Aerosil®). Preferably, silica is used with a specific surface area of 50 to 400 m²/g, determined by gas adsorption in accordance with Ph. Eur., 6th edition 2.9.26.

In a further embodiment, the oral dosage form, especially when present in tablet form, may, for example, additionally contain lubricant (b5). Lubricants (b5) are generally used in order to reduce sliding friction. In particular the intention is to reduce the sliding friction found during tablet pressing between the punch moving up and down in the die and the die wall, on the one hand, and between the edge of the tablet and the die wall, on the other hand. Suitable lubricants are, for example, stearic acid, adipic acid, sodium stearyl fumarate (Pruv®), magnesium stearate and/or calcium stearate.

The task of wetting agents (b6) is to improve the wettability of active agents and/or excipients. Preferred wetting agents (b6) herein are surfactants with a HLB value of 10 or more. Examples of these which can be mentioned are anionic, cationic, amphoteric or non-ionic surfactants. It is, for example, possible to use the following surfactants with an HLB value of 10, or representatives of the following classes of surfactants with an HLB value of 10 or more: polyoxyethylene fatty alcohol ether, e.g. macrogol lauryl ether, (e.g. Brij®, especially Brij® 56 and higher), ethoxylated sorbitan fatty acid ester (also known as polyoxyethylene sorbitan fatty acid ester, e.g. Tween®, especially Tween® 20, 21, 40, 60, 65, 80, 81), polyoxyethylene fatty acid glycerides, e.g. macrogol glycerine mono-fatty acid ester, such as macrogol 1000 glycerine mono-laurate, macrogol 1000 glycerine monostearate, macrogol 1000 glycerine mono-oleate, polyoxyethylene fatty acid ester, such as macrogol stearate 400, polyoxyl 40 stearate, polyoxyl 50 stearate, sucrose fatty acid ester, such as sucrose monooleate, sucrose monostearate, sucrose monomyristate, sucrose monopalmitate, non-ionic macromolecular surfactants, such as poloxamers, sodium lauryl sulphate (also known as sodium dodecyl sulphate), sodium cetyl stearyl sulphate, phospholipids, ethoxylated castor oil, soya lecithin and others, and also mixtures of two or more of the above-mentioned surfactants. Sodium lauryl sulphate is particularly preferred.

The statements made herein regarding the method of the invention and the process steps of the invention also apply, independently of the method, to the intermediate obtained or obtainable thereby and correspondingly also to the intermediate product, e.g. granules, or the oral dosage form. Similarly, statements made, with the exception of specifications of amounts and particle sizes concerning other ingredients contained in the intermediate and concerning the intermediate itself, also apply, mutatis mutandis, to the intermediate product of the invention, e.g. granules, or the oral dosage form of the invention.

Granules containing (a) fingolimod and (b) one or more pharmaceutically acceptable excipients are a further subject matter of the invention. The granules of the invention are obtainable by a method comprising the following steps:
(i) optionally mixing (a) fingolimod and (b) the excipient or the plurality of excipients,
(ii) jointly comminuting (a) fingolimod and (b) the one or more excipients into intermediate particles such that 90 per cent by volume of all the resulting intermediate particles have a particle size of less than 250 μm and greater than 0.6 μm.
(iii) granulating the intermediate and optionally one or more additional pharmaceutical excipients.

As far as steps (i) and (ii) are concerned, reference is made to the statements regarding the method of preparing the intermediate.

“Granulating” is generally understood to mean the formation of relatively coarse or granular aggregate material as a powder by assembling and/or aggregating finer powder particles (agglomerate formation, or build-up granulation) and/or the formation of finer granules by breaking up coarser aggregates (disintegration, or break-down granulation).

Granulation can conventionally mean wet or dry granulation. Dry granulation is generally carried out using pressure or temperature. Wet granulation (hereinafter used synonymously with moist granulation) is generally carried out using surface stabilisers (b2) and/or solvents or dispersants. Granulation is generally carried out in conventional granulating devices, such as extruder, perforated-disk, perforated-roll, or fluidised-bed granulators. Compulsory mixers or spray dryers can likewise be used.

The granulation time, especially in the case of wet granulation is usually 1 minute to 1 hour, preferably 2 minutes to 30 minutes. Dry granulation is usually carried out as a continuous process.

Preferred embodiments of dry and wet granulation will now be explained.

Dry Granulation:

Dry granulation is usually preferred if the intermediate has been milled in a dry state.

In this embodiment of step (iii) of the method of the invention, the intermediate of the invention from step (ii) is compacted into flakes. The compacting conditions in step (iii) are preferably selected such that the flakes have a density of 1.03 to 1.8 g/cm³, especially 1.05 to 1.7 g/cm³. The compacting is preferably carried out in a roll granulator. The rolling force is preferably 2 to 50 kN/cm, more preferably 4 to 30 kN/cm, especially 10 to 25 kN/cm. The gap width of the roll granulator is, for example, 0.8 to 5 mm, preferably 1 to 4 mm, more preferably 1.5 to 3 mm, especially 1.8 to 2.8 mm. After that, the flakes are preferably granulated.

The granulation can generally be performed with methods known in the prior art.

In a preferred embodiment, the granulation of the flakes is performed in a screen mill. In this case, the mesh width of the screen insert is usually 0.063 to 2 mm, preferably 0.5 to 1.5 mm, especially preferably 0.71 to 1.25 mm.

The resulting particles (granules) preferably have a D₅₀ value of 500 to 10 μm, more preferably 350 to 50 μm, and especially 250 to 60 μm. In the context of the present invention, the particle size of the granules is determined by means of screen analysis (preferably using a Retsch® AS 2000).

For the dry granulation process, substantially only the intermediate of the invention is used. Optionally, but not preferably, small amounts of pharmaceutical excipients can be added which are not present in the particle size distribution of the invention. Examples of these are flow conditioning agents. In the dry granulation step, 90 to 100% by weight, more preferably 95 to 99.9% by weight, of the intermediate of the invention are preferably used, based on the total weight of the substances used.

Wet Granulation:

Wet granulation can be performed with conventional methods. Wet granulation is preferred if the intermediate is prepared by means of wet-milling processes. Wet granulation is preferably carried out in a fluidised bed.

For this purpose, the intermediate from step (ii), preferably the moist intermediate from step (ii), is introduced into a fluidised bed.

For the wet granulation process, it is preferable that substantially only the intermediate of the invention is used. Optionally, but not preferably, small amounts of further pharmaceutical excipients can be added. In the wet granulation step, preferably 30 to 100% by weight, more preferably 95 to 99.9% by weight, even more preferably 70 to 99.0% by weight of the intermediate of the invention are used, based on the total weight of the substances used.

In a preferred embodiment, the wet granulation is carried out in a fluidised bed granulator, such as a Glatt® GPCG 3 (Glatt GmbH, Germany). The wet granulation can be performed using a class 3 dispersant or solvent, such as isopropanol, ethanol, a mixture of ethanol and water, aqueous solutions or pure water. The use of pure water is preferred here.

If in steps (ii) or (iii) the basic operations of wet granulation and/or wet milling are performed, it is normal to carry out a step of “drying”. The drying step can be performed after or at the same time as the granulation step.

“Drying” is understood for the purposes of this invention to mean the removal of liquids adhering to solids. Drying is generally performed in conventional drying apparatuses, such as cabinet or tray dryers, vacuum dryers, fluidised bed dryers, spray dryers or freeze dryers. The drying and granulation process is preferably performed in one and the same apparatus.

Intermediate particles, optionally with the addition of one or more further excipients (b), may also be spray-dried without wet granulation.

The drying conditions are preferably selected such that the content of dispersant in the resulting granules is 0.1 to 5% by weight. The content of residual dispersant is preferably 1 to 5,000 ppm, preferably 5 to 100 ppm.

In wet granulation, it is again preferable for a screen with a mesh width of 0.063 to 2 mm, preferably 0.5 to 1.5 mm, especially preferably 0.71 to 1.25 mm, to be used.

The particles (granules) resulting from the wet granulation step preferably have a D₅₀ value of 500 to 3 μm, more preferably 350 to 5 μm, and especially 250 to 10 μm. In the context of the present invention, the particle size of the granules is determined by means of laser diffractometry, as explained above with regard to particle size determination.

In addition, the granulation conditions in all the granulation processes are preferably selected such that the resulting granules have a bulk density of 0.2 to 0.85 g/ml, more preferably 0.3 to 0.8 g/ml, especially 0.4 to 0.7 g/ml. The Hausner factor is usually in the range from 1.03 to 1.3, more preferably from 1.04 to 1.20 and especially from 1.04 to 1.15. The “Hausner factor” in this context means the ratio of tapped density to bulk density. The bulk density and tapped density are determined in accordance with USP 24, test 616 “Bulk Density and Tapped Density”.

In addition, the mixing, milling and/or further processing conditions (e.g. granulation conditions) are preferably selected such that granules with a uniformity of the mixture of 90% to 110%, more preferably 92% to 108%, even more preferably 94% to 106%, particularly preferably 96% to 104% and especially 98% to 102%, are obtained. The “uniformity of the mixture” refers here to the uniformity of the content of active agent in different granule samples. In order to determine the uniformity of the mixture of the granules, 20 individual samples with a volume of 10 ml each are taken from the granules at random, and the uniformity of the content of active agent is determined as explained above. This means that each of twenty individual samples of the granules has a fingolimod content of between 90% and 110%, preferably 92% to 108%, even more preferably 94% to 106%, particularly preferably 96% to 104% and especially 98% to 102% of the average content of those twenty individual samples.

A method of preparing an oral dosage form containing (a) fingolimod and (b) one or more pharmaceutically acceptable excipients is a further subject matter of the invention. The method comprises the method of the invention of preparing the intermediate, and
(iii) optionally further processing the intermediate, optionally with the addition of one or more additional pharmaceutically acceptable excipients, such as by granulation, spray-drying or lyophilisation, into an intermediate product,
(iv) compressing the intermediate from step (ii) or the intermediate product from step (iii) and optionally one or more additional pharmaceutically acceptable excipients into tablets.
   or
   filling the intermediate from step (ii) or the intermediate product from step (iii) and optionally one or more additional pharmaceutically acceptable excipients into capsules or sachets or other suitable containers.

The intermediate product may be present, depending on the choice of the further processing step, in the form of, for example, granules, lyophilisate, spray-dried material or the like. Combinations of the above-mentioned further processing steps are also conceivable.

According to the present invention, an “intermediate product” is preferably understood to mean a pharmaceutical composition which is not administered directly. Embodiments are, however, also encompassed in which the intermediate product can be administered directly.

Depending on the configuration of the method of the invention, various possibilities are conceivable for steps (iii) and (iv), e.g.:

Embodiment 1: direct compression into tablets;

Embodiment 2: dry granulation and subsequent compression into tablets;

Embodiment 3: wet granulation and subsequent compression into tablets;

Embodiment 4: dry granulation and subsequent filling into dosage forms such as sachets, stickpacks or capsules;

Embodiment 5: wet granulation and subsequent filling into dosage forms such as sachets, stickpacks or capsules;

Embodiment 6: spray-drying and subsequent filling into dosage forms such as sachets, stickpacks or capsules;

Embodiment 7: spray-drying and subsequent compression into tablets;

Embodiment 8: lyophilisation and subsequent filling into dosage forms such as sachets, stickpacks or capsules;

Embodiment 9: lyophilisation and subsequent compression into tablets;

Embodiment 1 does not require a granulation step (iii), whereas embodiments 2 to 5 do. In the optional step (iii), the intermediate is therefore granulated.

In a preferred embodiment of the present invention, in step (iv) the intermediate from step (ii) or the granules from step (iii) are compressed into tablets.

The process of compressing can be carried out, as explained above, without further pre-treatment by compressing the intermediate from step (ii) (=direct compression) or after any further processing carried out in step (iii), e.g. granulation. Direct compression is preferred.

The tableting conditions here are preferably selected such that the resulting tablets have a tablet height to weight ratio of 0.004 to 0.02 mm/mg, more preferably 0.006 to 0.0018 mm/mg, particularly preferably 0.004 to 0.015 mm/mg.

The tableting machines used to produce the tablets can be conventional tableting machines. A rotary tableting press or eccentric press are preferably used. In the case of rotary tableting presses, a compressive force of 2 to 40 kN, preferably 2.5 to 35 kN, is usually applied. In the case of eccentric presses, a compressive force of 1 to 20 kN, preferably 2.5 to 10 kN, is usually applied. By way of example, the Korsch® EK0 is used.

In accordance with the invention, the resulting tablets preferably have a mass of 100 to 550 mg, such as 150 to 350 mg, 130 to 250 mg, 150 to 240 mg or particularly preferably 170 to 220 mg.

In the context of the invention, the resulting tablets may be coated or uncoated. In accordance with the invention, the film formers used for the coating process may preferably be cellulose derivatives, such as methyl cellulose (MC), ethyl cellulose (EC), hydroxyethyl cellulose (HEC), methacrylic acid/acrylate co-polymers, such as methacrylic acid/ethacrylate copolymer or methacrylic acid/methyl methacrylate copolymer, vinyl polymers, such as polyvinyl pyrrolidone or polyvinyl acetate phthalate or natural film formers, such as shellack. The coating preferably does not contain any active agent.

The thickness of the coating is usually 0.1 to 100 μm, preferably 1 to 80 μm.

It is preferable for the optionally applied film to have substantially no effects on the release. These are therefore preferably films with no influence on the release of the active agent. In the context of this invention, it is preferable for neither enteric film coatings nor delayed-release coatings to be used.

For the purposes of the invention, the resulting tablets should preferably exhibit a high level of hardness and low friability.

The resulting tablets preferably have a hardness of 50 to 300 N, particularly preferably 80 to 250 N, especially 100 to 220 N. The hardness is determined in accordance with Ph. Eur. 6.0, section 2.9.8.

In addition, the resulting tablets preferably have a friability of 0.1 to 0.8%, preferably 0.2 to 0.6% and particularly preferably 0.3 to 0.5%. The friability is determined in accordance with Ph. Eur. 6.0, section 2.9.7.

It has been shown that the intermediates of the invention are suitable for serving both as a basis for a dosage form with immediate release (or “IR” for short) and also with modified release (or “MR” for short).

In the case of an IR formulation, the release profile of the tablets of the invention according to the USP method (USP basket apparatus, 500 ml test medium; 0.1 N HCl and 0.2% sodium dodecyl sulfate, 37° C. and 100 rpm) after 10 minutes usually indicates a content released of at least 30%, preferably at least 60%, especially at least 98%.

Alternatively and/or at the same time, the tablets of the invention are preferably ones that disintegrate at a moderate speed. In the case of an IR formulation, according to the USP method (USP basket apparatus, 500 ml test medium; 0.1 N HCl and 0.2% sodium dodecyl sulfate, 37° C. and 100 rpm) after 10 minutes, the tablet has a content released of, for example, no more than 98%, preferably no more than 90%, especially no more than 75%.

In the case of an MR formulation, the release profile of the tablets of the invention according to the USP method (USP basket apparatus, 500 ml test medium; 0.1 N HCl and 0.2% sodium dodecyl sulfate, 37° C. and 100 rpm) after 60 minutes usually indicates a content released of 10, preferably 20, especially 30%.

Alternatively and/or at the same time, the tablets of the invention are preferably ones that disintegrate at a moderate speed. In the case of an MR formulation, according to the USP method (USP basket apparatus, 500 ml test medium; 0.1 N HCl and 0.2% sodium dodecyl sulfate, 37° C. and 100 rpm) after 10 minutes, the tablet has a content released of, for example, no more than 98%, preferably no more than 90%, especially no more than 75%.

The above details regarding hardness, friability, content uniformity and release profile preferably relate herein to the non-film-coated tablet for an IR formulation. For a modified release tablet, the release profile relates to the total formulation.

In a further embodiment of the present invention, in step (iv) the intermediate from step (ii) or the intermediate product, e.g. granules, from step (iii) are filled into dosage forms, such as sachets, stickpacks or capsules.

A further subject matter of the present invention is accordingly an oral dosage form containing the intermediate of the invention and/or the granules or intermediate product of the invention. Furthermore, the subject matter of the invention includes an oral dosage form which is obtainable by a method of the invention of preparing the oral dosage form.

An oral dosage form for the purposes of the invention is understood to mean a drug formulation which is applied orally. Oral dosage forms in the context of this invention are preferably tablets or capsules, particularly preferably tablets. Alternatively, sachets or stickpacks containing the intermediate of the invention (optionally in granulated form) may also be regarded as oral dosage forms. In addition to a homogeneous distribution of the active agent, the oral dosage forms of the invention have the advantage of good storage stability.

The intermediate accordingly contains the intermediate product, e.g. granules, and the oral dosage form of the present invention preferably contains no further active agent. Embodiments with further active agents are, however, also conceivable.

According to a preferred embodiment, the uniformity of the content of fingolimod (a) in the oral dosage form of the invention is characterised by the fact that each of ten dosage form units has a fingolimod content of between 90% and 110%, preferably 92% to 108%, even more preferably 94% to 106%, particularly preferably 96% to 104% and especially 98% to 102% of the average content of those ten dosage form units. The “uniformity of the content of fingolimod (a)” is determined here in accordance with Pharm. Eur. 2.9.6.

In particularly preferred embodiments, fingolimod is contained in the oral dosage form in amounts of 0.5 mg, 0.75 mg, 1 mg, 1.25 mg, 1.5 mg, 1.75 mg, 2 mg or 2.5 mg.

Finally, an oral dosage form for the treatment of multiple sclerosis, preferably relapsing-remitting multiple sclerosis, is also a subject matter of the present invention.

The examples provided here for the excipients are optional, i.e. they may be used in the intermediates, intermediate products, such as granules, and dosage forms of the invention, but embodiments are of course also encompassed which are free of one or more of the substances or combinations of substances mentioned as examples in each case.

The invention will now be explained with reference to the following examples.

### EXAMPLES

### Example 1

### Preparation of an Intermediate by Means of Dry Milling

The following substances were used to prepare an intermediate by means of dry milling.

0.63 g fingolimod hydrochloride was milled for 5 minutes together with 16.89 g sucrose (dry) in an air jet mill (Alpine Jet Mill). After that, 22.52 g HPMC was added and milled again for 5 minutes. In two further steps, in total 129.36 g sucrose was added and milled again for 5 minutes in each case. This resulted in an intermediate with particularly advantageous homogeneity.

The intermediate was filled into capsules, each of them having the following composition:

| | | |
|---|---|---|
| fingolimod HCl | 0.56 | mg |
| sucrose | 130 | mg |
| hydroxypropyl methyl cellulose (HPMC) | 20 | mg |

### Example 2

### Preparation of a Tablet Proceeding From an Intermediate According to Example 1

The intermediate of Example 1 was mixed with 78.75 g Avicel® 101, 9.0 g sodium carboxymethyl starch and 4.5 g Aerosil® for 20 minutes in a free-fall mixer (Turbula TB 10). 2.25 g Magnesium stearate was added to the resulting mixture through a 0.5 mm screen and the mixture resulting then was mixed for 3 minutes. After that, the mixture was compressed into a tablet using an eccentric press (Korsch), each tablet having the following composition:

| | | |
|---|---|---|
| fingolimod HCl | 0.56 | mg |
| sucrose | 130 | mg |
| hydroxypropyl methyl cellulose (HPMC) | 20 | mg |
| Avicel ® 101 (microcrystalline cellulose) | 70 | mg |
| sodium carboxymethyl starch | 8 | mg |
| Aerosil ® (colloidal silica) | 4 | mg |
| magnesium stearate | 2 | mg |

### Example 3

### Preparation of an Intermediate be Means of Wet Milling

The following substances were used to prepare an intermediate by means of wet milling:

| | | |
|---|---|---|
| fingolimod HCl | 0.5 | g |
| Povidon ® 25 | 0.5 | g |
| sodium lauryl sulphate | 0.05 | g |

Fingolimod was milled for an hour together with Povidon® 25 and sodium lauryl sulphate in dispersant in a Netzsch MicroCer to form an intermediate.

### Example 4

### Preparation of a Tablet Proceeding from an Intermediate According to Example 3

10 g microcrystalline cellulose were added to the resulting suspension containing the intermediate of Example 3, and this was spray-dried on a Büchi spray tower.

The intermediate product obtained was mixed for 25 minutes with 75 g silicified microcrystalline cellulose, 10 g Kollidon CL, 2.0 g colloidal silica in a free-fall mixer (Turbula TB 10). After that, 1.0 g magnesium stearate was added through a 0.5 mm screen and mixed again for 3 minutes. After that, the resulting mixture was compressed into a tablet on a Korsch eccentric press EK0, each tablet having the following composition:

| | | |
|---|---|---|
| fingolimod HCl | 1 | mg |
| Povidon ® 25 | 1 | mg |
| sodium lauryl sulphate | 0.1 | mg |
| microcrystalline cellulose | 20 | mg |
| silicified microcrystalline cellulose | 150 | mg |
| Kollidon ® CL | 20 | mg |
| colloidal silica | 4 | mg |
| magnesium stearate | 2 | mg |

1. A method of preparing an intermediate comprising (a) fingolimod and (b) one or more pharmaceutically acceptable excipients, comprising the steps of:
(i) optionally mixing (a) fingolimod and (b) the excipient or the plurality of excipients,
(ii) jointly comminuting (a) fingolimod and (b) the one or more excipients into intermediate particles such that 90 per cent by volume of all the resulting intermediate particles have a particle size of less than 250 μm and greater than 0.6 μm.

2. The method of preparing an intermediate as claimed in claim 1, wherein the joint comminution (ii) comprises joint milling.

3. The method of preparing an intermediate as claimed in claim 1, wherein the joint comminution (ii) is carried out in such a way that the resulting intermediate particles have a monomodal particle size distribution.

4. The method of preparing an intermediate as claimed in claim 1, wherein the joint comminution (ii) is carried out in such a way that 50 per cent by volume of all the resulting intermediate particles have a particle size of 80 μm or less:
D₅₀≦80 μm.

5. The method of preparing an intermediate as claimed in claim 1, wherein the joint comminution (ii) is carried out in such a way that the particle sizes of 90 per cent by volume of all the resulting intermediate particles (D₉₀), the particle sizes of 50 per cent by volume of all the resulting intermediate particles (D₅₀) and the particle sizes of 10 per cent by volume of all the resulting intermediate particles (D₁₀) satisfy the following relationship:
(D₉₀−D₁₀)/D₅₀≦7

6. The method of preparing an intermediate as claimed in claim 1, wherein the pharmaceutically acceptable excipient or the plural pharmaceutically acceptable excipients (b) comprise at least one of the following excipients: (b1) filler, (b2) surface stabiliser, (b4) flow conditioning agent and/or (b6) wetting agent.

7. An intermediate comprising (a) fingolimod and (b) one or more pharmaceutically acceptable excipients, prepared by the method as claimed in claim 1.

8. An intermediate comprising particles of (a) fingolimod and (b) one or more pharmaceutically acceptable excipients, wherein 90 per cent by volume of the particles have a particle size of less than 250 μm and greater than 0.6 μm.

9. The intermediate as claimed in claim 8, wherein the particles have a monomodal particle size distribution.

10. The intermediate as claimed in claim 8, wherein 50 per cent by volume of the particles have a particle size of 80 μm or less:
D₅₀≦80 μm.

11. The intermediate as claimed claim 8, wherein the particle sizes of 90 per cent by volume of the particles (D₉₀), the particle sizes of 50 per cent by volume of the particles (D₅₀) and the particle sizes of 10 per cent by volume of the particles (D₁₀) satisfy the following relationship:
(D₉₀−D₁₀)/D₅₀≦7

12. Granules comprising (a) fingolimod and (b) one or more pharmaceutically acceptable excipients, obtainable by the method of preparing an intermediate as claimed in claim 1, and the following step:
(iii) granulating the intermediate and optionally one or more additional pharmaceutically acceptable excipients.

13. A method of preparing an oral dosage form comprising (a) fingolimod and (b) one or more pharmaceutically acceptable excipients, comprising the method of preparing the intermediate as claimed in claim 1, and the following steps:
(iii) optionally further processing the intermediate, optionally with the addition of one or more additional pharmaceutically acceptable excipients, by granulation, spray-drying or lyophilisation, into an intermediate product,
(iv) compressing the intermediate from step (ii) or the intermediate product from step (iii) and optionally one or more additional pharmaceutically acceptable excipients into tablets,
or
filling the intermediate from step (ii) or the intermediate product from step (iii) and optionally one or more additional pharmaceutically acceptable excipients into capsules or sachets or other suitable containers.

14. An oral dosage form comprising (a) fingolimod and (b) one or more pharmaceutically acceptable excipients, obtainable by the method as claimed in claim 13.

15. An oral dosage form comprising the intermediate as claimed in claim 12.

16. The oral dosage form as claimed in claim 14, wherein the uniformity of the content of fingolimod, determined in accordance with Ph. Eur. 2.9.6, is characterised in that each of ten dosage form units has a content of fingolimod which lies between 90 and 110% of the average content of those ten dosage form units.

17. The oral dosage form as claimed in claim 14, wherein the oral dosage form is a tablet form.

18. The oral dosage form as claimed in claim 14, for the treatment of multiple sclerosis.
